(19) United States
(12) Patent Application Publication
(10) Pub. No.: US 2014/0031698 A1
(43) Pub. Date: Jan. 30, 2014

(54) APPARATUS AND METHOD FOR SENSING BONE POSITION AND MOTION

(71) Applicant: San Diego State University Research Foundation, (US)

(72) Inventors: Kee S. MOON, San Diego, CA (US); Yusuf OZTURK, San Diego, CA (US)

(21) Appl. No.: 13/875,701

(22) Filed: May 2, 2013

Related U.S. Application Data
(60) Provisional application No. 61/641,729, filed on May 2, 2012.

(51) Int. Cl. A61B 5/11; A61B 5/00

(52) U.S. Cl. CPC A61B 5/1126; A61B 5/0077
USPC 600/476

(57) ABSTRACT

This invention describes a novel photoplethysmographic system captures a joint shape change representing different positions of bones and tendons near the joint. At least one light source illuminates a joint interface and the light reflected from the joint location is captured by a photodiode sensor. Motions and positions of the bones surrounding the joint can be determined. One joint includes the knuckle. Finger lift-up motions, finger put-down motions, and finger bending events can be determined by monitoring a sensing area at the knuckle joint.

### CROSS-REFERENCE TO RELATED APPLICATION

This application claims the benefit under 35 U.S.C. §119(e) of U.S. Provisional Patent Application No. 61/641,729, filed 2 May 2012, and which application is incorporated herein by reference.

### TECHNICAL FIELD

Various embodiments described herein relate to an apparatus and method for sensing finger motion. More specifically, the embodiments relate to a wireless photoplethysmographic knuckle sensor for capturing finger motion.

### BACKGROUND OF THE INVENTION

Sensing and capturing information on the motion of one or more human fingers has many applications. Finger or digit motion sensing is useful medical devices, computer input devices, electronic communication devices, gaming and entertainment devices, many other devices spanning many fields of technology. A number of commercial and laboratory devices have been developed to capture finger motions using various methods using various technologies such as fiber optic technology, acoustic technology, magnetic sensing technology, strain gauge technology, and electromagnetic technology.

### SUMMARY OF THE INVENTION

This invention describes a novel photoplethysmographic system captures a knuckle joint shape change representing finger lift-up and finger put-down motions over a sensing area and produces an optical intensity signal for use in detecting a finger bending event. At least one light source illuminates a knuckle joint interface and the light reflected from the knuckle joint location is captured by a photodiode sensor.

### BRIEF DESCRIPTION OF THE DRAWINGS

The embodiments will be readily understood by the following detailed description in conjunction with the accompanying drawings, wherein like reference numerals designate like structural elements, and in which:

FIG. 1 is a top view of a finger motion capture device for sensing finger motions, according to an example embodiment.

FIG. 2A is a side view of an optical photoplethysmographic knuckle motion sensor positioned to detect finger motion, according to an example embodiment.

FIG. 2B is a top view of an optical photoplethysmographic knuckle motion sensor positioned to detect finger motion, according to an example embodiment.

FIG. 3 is a perspective view of an optical photoplethysmographic knuckle motion sensor positioned to measure the change in knuckle shape including synovial fluid volume and the associated extensor tendon travel and the bone movement, according to an example embodiment.

FIG. 4 shows a system having a number of applications for an optical photoplethysmographic knuckle motion sensor applications, according to an example embodiment.

FIG. 5 shows a wireless optical photoplethysmographic knuckle motion sensor social network application, according to an example embodiment.

FIG. 6A shows a shows a circuit diagram of 2-channel signal amplifier for the wireless optical photoplethysmographic knuckle motion sensor, according to an example embodiment.

FIG. 6B shows a a 4 channel knuckle sensor utilizing analog Mux/DeMux to reduce circuit size, according to an example embodiment.

FIG. 7 shows a printed circuit board of 2-channel signal amplifier for the wireless optical photoplethysmographic knuckle motion sensor, according to an example embodiment. FIG. 7 correlates to the circuit diagram shown in FIG. 6A above.

FIG. 8 shows an assembly of the wireless optical photoplethysmographic knuckle motion sensor installed for wireless finger motion sensor with robotic rehabilitation device, according to an example embodiment.

### DETAILED DESCRIPTION

In the following paper, numerous specific details are set forth to provide a thorough understanding of the concepts underlying the described embodiments. It will be apparent, however, to one skilled in the art that the described embodiments may be practiced without some or all of these specific details. In other instances, well known process steps have not been described in detail in order to avoid unnecessarily obscuring the underlying concepts.

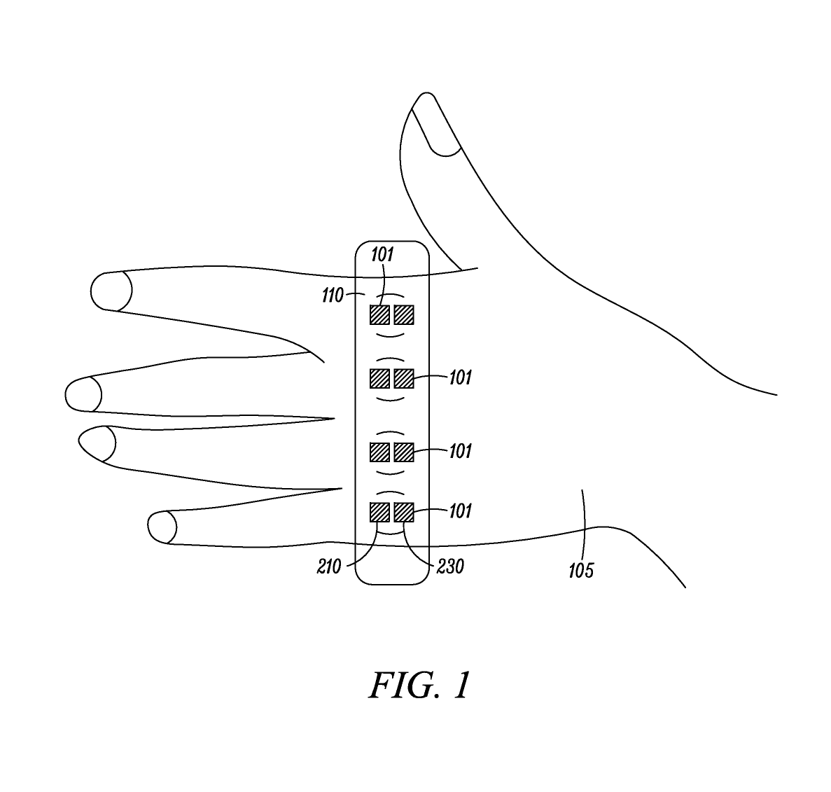

FIG. 1 is a top view of a wearable finger motion capture device 100 device for sensing finger motions on a hand 102 using at least one optical photoplethysmographic motion sensor 101, according to an example embodiment. The finger motion capture device 100 is a wearable device. As shown in FIG. 1, the finger motion capture device 100 includes a strap 110 having a plurality of optical photoplethysmographic sensors 101 attached thereto. The optical photoplethysmographic sensors 101 are positioned on the strap 110 to correspond to the position of at least one knuckle, namely, a prominence of the dorsal aspect of a joint of a finger, especially of one of the joints that connect the fingers to the hand. More specifically, the knuckle is the dorsal aspect of any interphalangeal joint, but especially of the metacarpophalangeal joints of the flexed fingers. As shown in FIG. 1, the finger motion capture device 100 includes photoplethysmographic sensors 101 positioned on the strap 110 at each of four knuckles (not shown in FIG. 1) of the hand 105. It is contemplated that different people will have hands 105 of different sizes and that there may be different spacings between the knuckles on specific hands 105. It is contemplated, in one embodiment, that there are different sized straps 110 having different spacings between the photoplethysmographic sensors 101. In another embodiment, the photoplethysmographic sensors 101 are adjustable with respect to the strap 110 so as to accommodate different spacings between the knuckles of a particular hand 105. Each of the photoplethysmographic sensors 101 includes a light source 210 and a light detector 230.

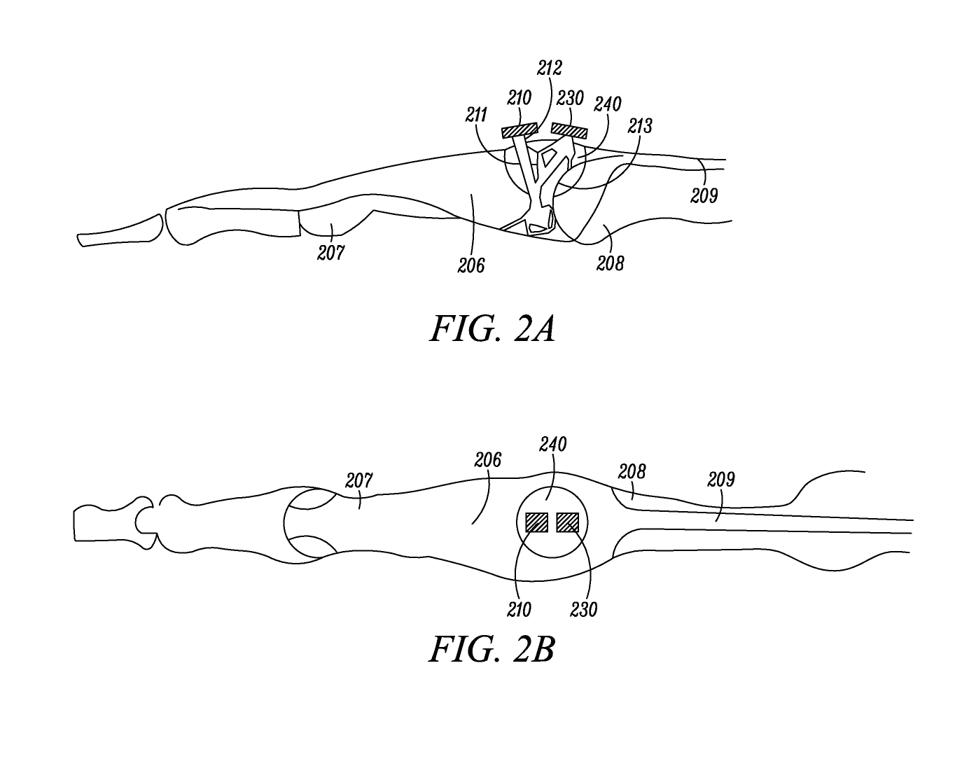

FIG. 2A is a side view and FIG. 2B is a top view of an optical photoplethysmographic sensor positioned on a knuckle to detect finger motion, according to an example embodiment. Now referring to FIGS. 2A and 2B, the optical photoplethysmographic sensor 101will be further discussed. As mentioned above, the photoplethysmographic sensors 101 includes the light source 210 and the light detector 230. Light is emitted from the light source 210. Reflected light is also received or gathered at the light detector 230. The emission of light from the light emitter 210 is depicted by arrows 211 and 212. The reflected light from the knuckle joint is depicted by arrow 213. The reflected light 213 strikes the light detector 230. The light detector is, in one embodiment, a photodetector which produces a signal in response to an amount of light striking the light detector 230. The light from the light emitter 210 illuminates at least a portion of the joint associated with the knuckle. The light illuminated area within or around the knuckle joint is depicted by the circle 240. It should be noted that the light illuminated area 240 can be larger or smaller depending upon the size of the knuckle joint as well as the amount of illumination provided by the light emitter 210. Also shown in FIGS. 2A and 2B, are the phalanges 207, the metacarpals 208, the exterior tendon 209 and the extensor hood 206.

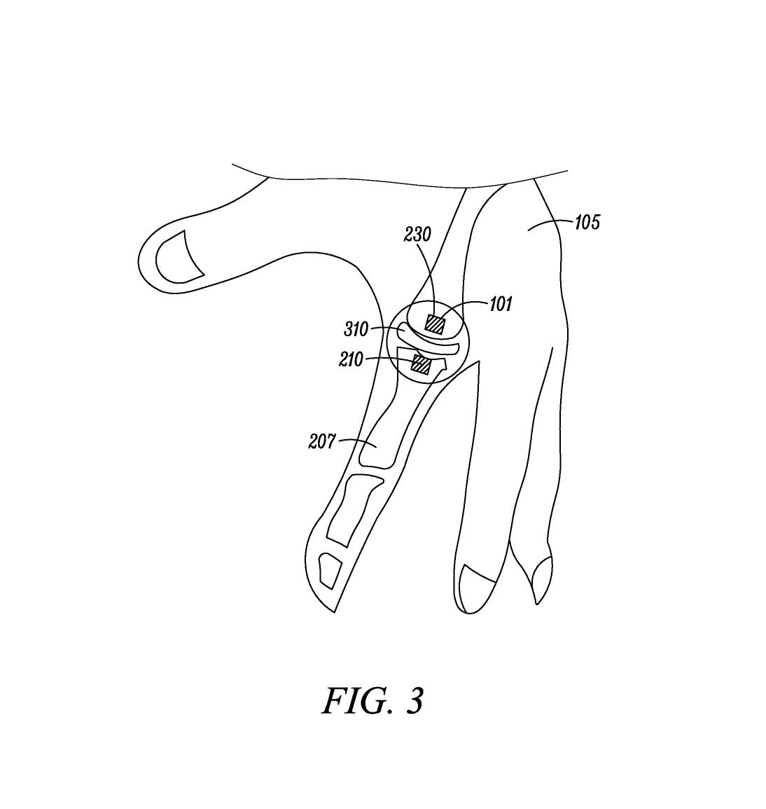

FIG. 3 is a perspective view of an optical photoplethysmographic sensor 101 positioned to measure the change in knuckle shape, according to an example embodiment. FIG. 3 is an x-ray view of the hand and specifically of one knuckle joint of the hand 105. Now looking at FIGS. 2A, 2B and 3, it can be seen that the knuckle joint includes a sack of synovial fluid 310 and several tendons the pass through the joint to connect the metacarpals 208 to the phalanges 207. The sensor 101 measures the variations of the reflected optical intensity that originate from the light absorption caused from shape changes of finger knuckles. More specifically, as the finger is moved there is a change in shape in the knuckle joint that results in different amounts of light being reflected back to the light detector 230 of the sensor 101. In other words, the sensor 101 measures motion of each finger by measuring the amount of light reflected from the knuckle which corresponds to an associated knuckle shape (or joint volume) change caused by different finger positions with respect to the hand. The main knuckle joints are formed by the connections of the phalanges 207 to the metacarpals 208. The knuckle joints work like a hinge when fingers 207 bend and straighten. Therefore, a click finger motion causes tendon 209 travel and knuckle bone position and synovial fluid 310 volume changes as a function of finger angles. The entire motion can result in a signal that really is a signature of a click motions. Other motions can have other signatures.

The change in knuckle shape, including synovial fluid volume 310 that is caused by a finger motion and the associated extensor tendon 209 travel and the bone movement can be detected by illuminating the knuckle joint location with the light from the light source 210, such a light-emitting diode (LED), and then measuring the amount of light reflected to a light detector 230, such as a photodiode. This can be done at one knuckle or several knuckles. The light detector 230 produces a signal based on the amount reflected light received at the light detector 230. This can be correlated to a finger position with respect to the hand or main portion of the hand 105. This can be done for each joint or knuckle joint on the hand 105. The signal produced by the light detector 230 can be sent to a processor to determine the finger position of a particular joint. The processor can be a computer, a microprocessor or any other type of processor. The knuckle motion sensor 100 can be wired to provide a hardwired connection to the processor. In the embodiment shown in FIG. 1, the knuckle motion sensor 100 communicates wirelessly with a processor or other processing unit. The result is that the knuckle motion sensor 100 does not inhibit the wearer of the device as much as a device which is hardwired to computer or other processor. In addition, the knuckle motion sensor 100 is not connected or attached or otherwise associated with individual fingers or digits. This allows the user to have much more freedom of motion when partaking in various activities which require the use of the fingers or digits. The knuckle motion sensor 100 may also be termed as an optical photoplethysmographic knuckle motion sensor.

The optical photoplethysmographic knuckle motion sensor device 100 measures the reflective optical density in a knuckle joint by emitting and gathering a light source. It is recognized that the finger knuckles are playing an important role in motion intention sensing of independent finger motions. Therefore measuring knuckle activation is important to understanding finger motion without any finger attachment devices that can potentially cause hampering sophisticated finger motions such as playing instruments.

The wireless photoplethysmographic knuckle sensor device 100 provides a for a portable, lightweight and easy to wear finger motion capture device with low noise. The sensor measures the variations of the reflected optical intensity that originate from the light absorption caused from shape changes of finger knuckles. One or multiple-sensor attachment measures motion of each finger from associated knuckle shape (or joint volume) change. The main knuckle joints are formed by the connections of the phalanges to the metacarpals. The knuckle joints work like a hinge when fingers bend and straighten. Therefore, a click finger motion causes tendon travel and knuckle bone position and synovial fluid volume changes as a function of finger angles.

The change in knuckle shape including synovial fluid volume that is caused by a finger motion and the associated extensor tendon travel and the bone movement can be detected by illuminating the knuckle joint location with the light from a light-emitting diode (LED) and then measuring the amount of light reflected to a photodiode (FIG. 3). Wirelessly transmitting these finger motion data to the various electronics such as a computer for input information process can generate a number of new applications.

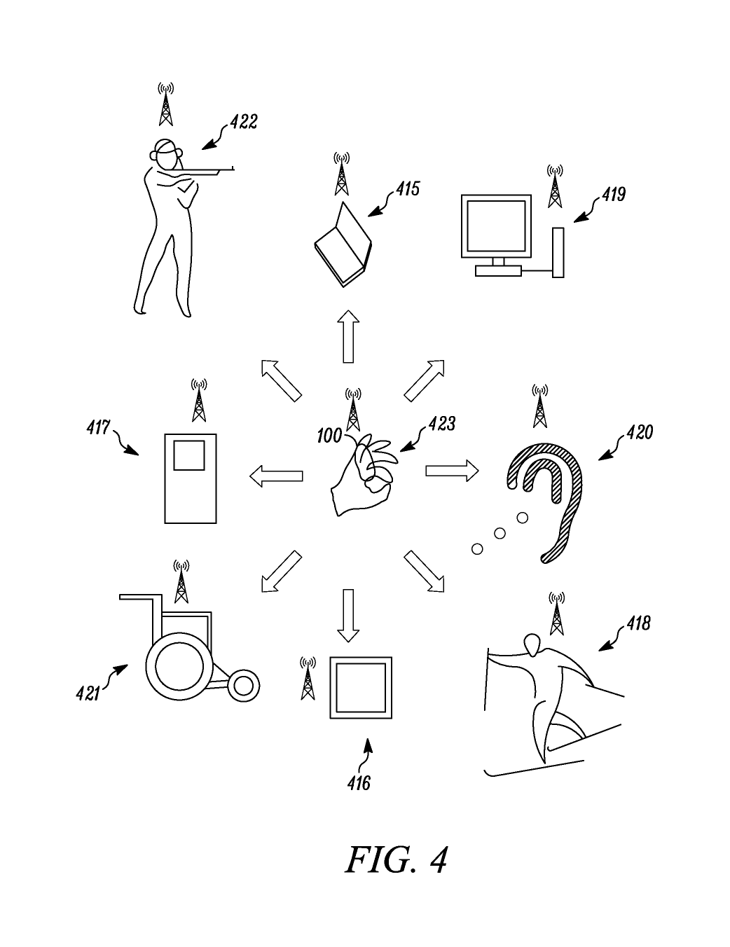

FIG. 4 shows a system having a number of applications for an optical photoplethysmographic knuckle motion sensor applications, according to an example embodiment. The new applications are as described in the following paragraphs.

Application 1: Wireless Computer Input Device

A wireless device knuckle sensor can produce signals that replace conventional cmputer keyboard. By monitoring moving fingers as they move to input various letters, numbers and signal, the physical keyboard can be removed. The wireless device can be used as a computer input device 415 that allows the user to enter characters or commands formed by simply moving one or more fingers like playing a piano. For example, five knuckles in one hand can generate (theoretically) 120 different combinations of signals. With a multi-axis accelerometer, the number of signals can be multiplied if necessary. Particularly, the device can enter a large number of combinations of text or commands (including mouse motion) to a small-size computer 419 such as a cell phone 417 that is too small to contain a normal-sized keyboard and a mouse. Since the device can be operated typically with one hand without actual keyboards, therefore, it provides a hand and eye coordination free computer input environment. In a cell phone 417, the screen is also used as an input device where a keyboard is displayed on part of the screen. The screen size however is not large enough and requires the keypad to be divided into several screens. In addition the small screen size makes data entry a slow process. The closeness of the characters also makes data entry on a small screen size a challenge for people with large fingers. The technology presented herein provides an answer to many of the problems associated with cell phone 417 data entry. It will also enable a larger penetration of cell phones 417 as a data collection tool in environments where a smaller screen is not usable (such as cold places where people wear gloves). It should be noted that this same technology can be used to free desk space in an office environment. In one embodiment, the combination of different knuckle intensities measured at one instant can indicate the character, number or symbol being input. In another embodiment, the different intensities over a portion of time produce a signature signal that indicates input of a particular letter, symbol or number.

The knuckle sensor technology presented here will enable cell phone applications that are limited now due to the difficulty of entering data using traditional means on a cell phone 417 or tablet device. The technology will also improve usability of cell phone 417 or tablet devices 416 as a general purpose communication and computing device by increasing the rate of entering data through different finger movements and combination of finger motion with other sensor data.

Application 2: Wireless Gaming Input Device

A wireless device knuckle sensor can replace conventional gaming input device 219 that is used with games or entertainment systems 219 to provide input to a video game, typically to control an object or character in the game. Signals produced by the knuckle sensor from moving fingers and a moving hand can substitute for a game controller that requires certain knobs or buttons or joysticks and the like to be pushed or otherwise moved to provide input to a game. The wireless device as a game controller for computer and video games can achieve greater speed and accurate movement for the gamer. Furthermore, it provides a large number of gaming signal input combinations that would be useful for complex gaming software that requires various gaming inputs from gamers. For example, five knuckles in one hand can generate at least 120 different combinations of signals. With a multi-axis accelerometer, the device can detect the game player's motions and finger motions as the inputs for a game. In one embodiment, the device can be operated with one hand without holding an actual device. This would provide a hand and eye coordination free gaming input environment. In another embodiment, two handed control oculd be used to add further input and allow for still more complex control. It is further contemplated that certain movement over time could produce signature signals for controlling a game or the like. It can also be used as a communication device in a gaming application where participants use sign language 420 to communicate ideas with each other.

Application 3: Wireless Sign Language Translation Device

With a multi-axis accelerometer, the wireless knuckle sensor device can detect the user's hand/arm motions and finger motions as a communication aid for the deaf or people who are hard of hearing. A wireless device knuckle sensor with a computer-based system can convert the sign language motions of individual speech to text or computer-generated voice (FIG. 4). The wireless devices can provide instantaneous translation of sign language. The device can be used as a means of communication in dark or noisy areas by exchanging signs and translating these signs into messages by means of an actuator at the receiving end (FIG. 4). In education domain, it can be used as a teaching appliance for teaching sign language or as a self assessment device for one to learn sign language themselves.

### Application 4: Remote Control and Rehabilitation

Many people suffer from diseases that limit their bodily functions. In such cases, this device is an effective rehabilitation device for a number of patients who have a disorder of the body's nervous system. This technology can be used in military and industrial applications for remote control of robots, vehicles and appliances (FIG. 4). It can be used by physically disabled people to control appliances and robots around them to aid their mobility or interactions with real and virtual environments.

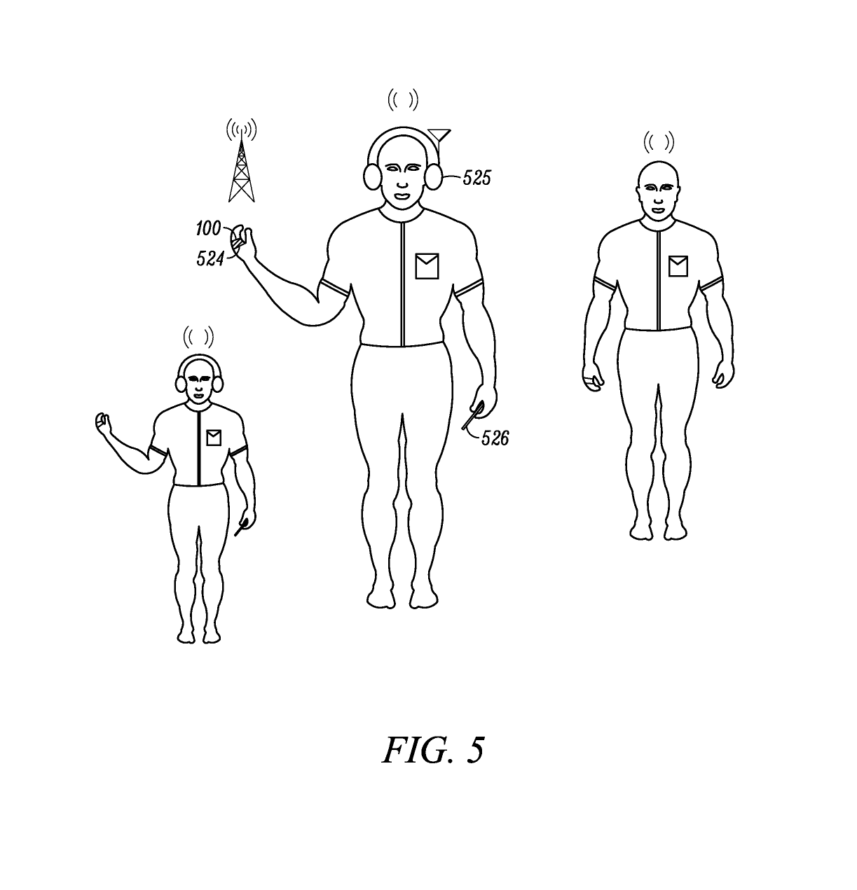

FIG. 5 shows a wireless optical photoplethysmographic knuckle motion sensor social network application, according to an example embodiment. This device will enable people with certain disabilities become an active participant of social networks and environments as a communication enables between people with disabilities and without (FIG. 5). In social gaming applications it can be used for silent chatting or silent games. In a gaming application where the shape of the hand (such as flat hand or fist) makes a difference (such as sports gaming, exercise and other applications requiring hand shape interaction) the technology can offer a solution to detect the shape of the hand and communicate to a game console or computer.

Various example embodiments include the following:

1. An optical photoplethysmographic knuckle motion sensor which measures the reflective optical density in a knuckle joint by emitting and gathering a light source.

2. A sensor that measures the variations of the reflected optical intensity that originate from the light absorption caused from shape changes of finger knuckles.

3. The measurement of the change in knuckle shape including synovial fluid volume that is caused by a finger motion and the associated extensor tendon travel and the bone movement by illuminating the knuckle joint location with the light from a light-emitting diode (LED) and then measuring the amount of light reflected to a photodiode.

4. A number of new commercial applications by wirelessly transmitting finger motion data to the various electronics such as a computer for input information process.

5. A device that can enter a large number of combinations of text or commands (including mouse motion) to a small-size computer such as a cell phone or tablet computer that is too small to contain a normal-sized keyboard and a mouse.

6. A device can be operated with one hand without actual keyboards, therefore, it provides a hand and eye coordination free computer input environment.

7. A device that provides a large number of gaming signal input combinations that would be useful for complex gaming software that would require various gaming inputs from gamers.

8. An effective rehabilitation device for a number of patients who have a disorder of the body's nervous system.

9. A computer-based system that can convert the sign language motions of individual speech to text or computer-generated voice, and thus provides instantaneous translation of sign language.

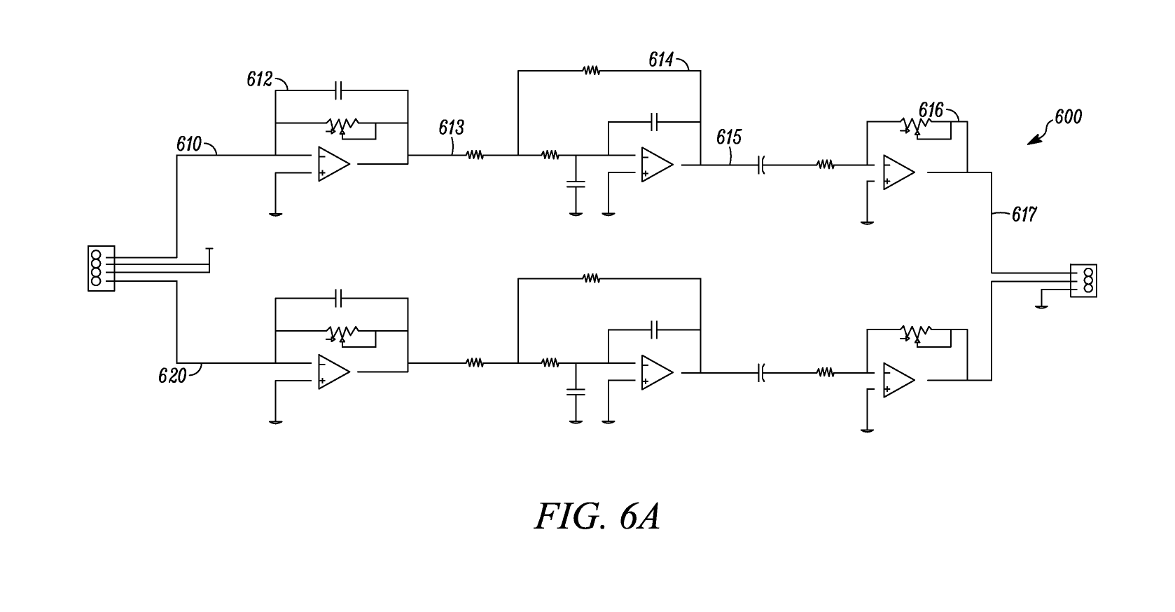

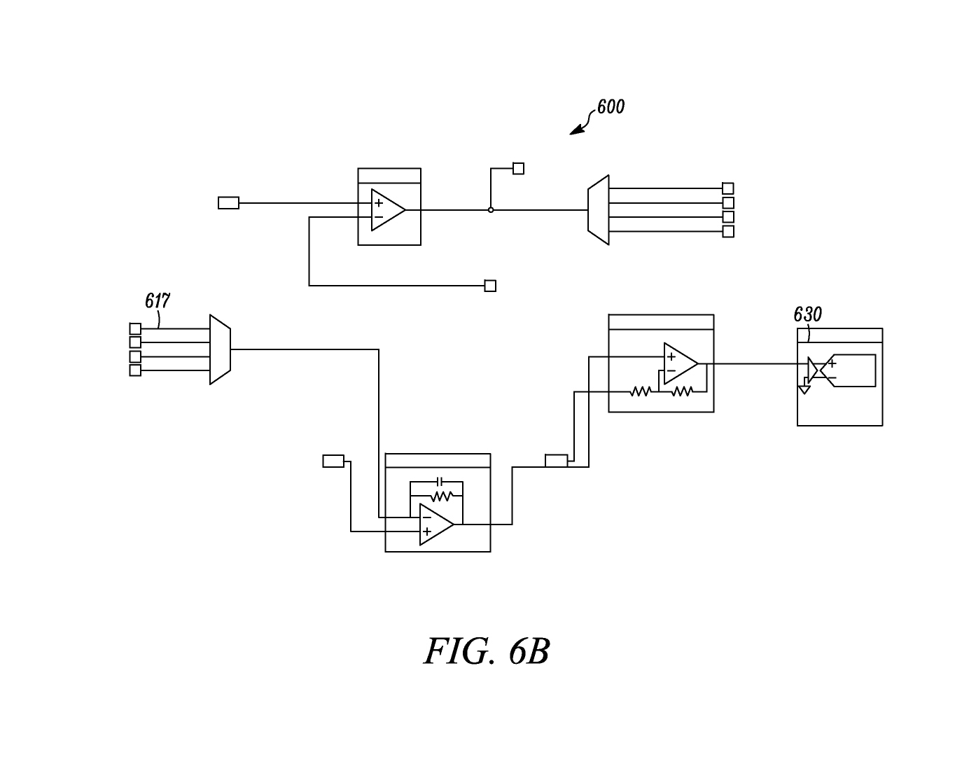

FIG. 6A shows a shows a circuit diagram 600 of 2-channel signal amplifier for the wireless optical photoplethysmographic knuckle motion sensor, according to an example embodiment. A first channel is depicted by the reference number 610 and the second channel is depicted by the reference number 620. Each of the first channel 610 and the second channel 620 have substantially identical components. Therefore, for the sake of brevity only the first channel 610 and it's components will be discussed. The circuit 600 includes a preamplification portion 612, a signal filtering portion 614 and power amplification portion 616. The preamplifier portion 612 amplifies the signal received from a photodetector of the device 100. The amplified signal 613 is input to the filter to remove unwanted noise and enhance the signal. The filtered output 615 is then input to the power amplification portion. The output 617 from the power amplification portion 616 is input to the next portion of the circuit shown in FIG. 6B.

FIG. 6B shows a 4 channel knuckle sensor utilizing analog Mux/DeMux to reduce circuit size, according to an example embodiment. This is a continuation of the circuit 600 shown n FIG. 6A. The circuit 600 also includes an analog to digital converter 630. The output is sent wirelessly to a computing device where the signal is compared to past signals and correlated to either a position of one or more knuckles or to a motion of one or more bones, such as fingers and those of the hand.

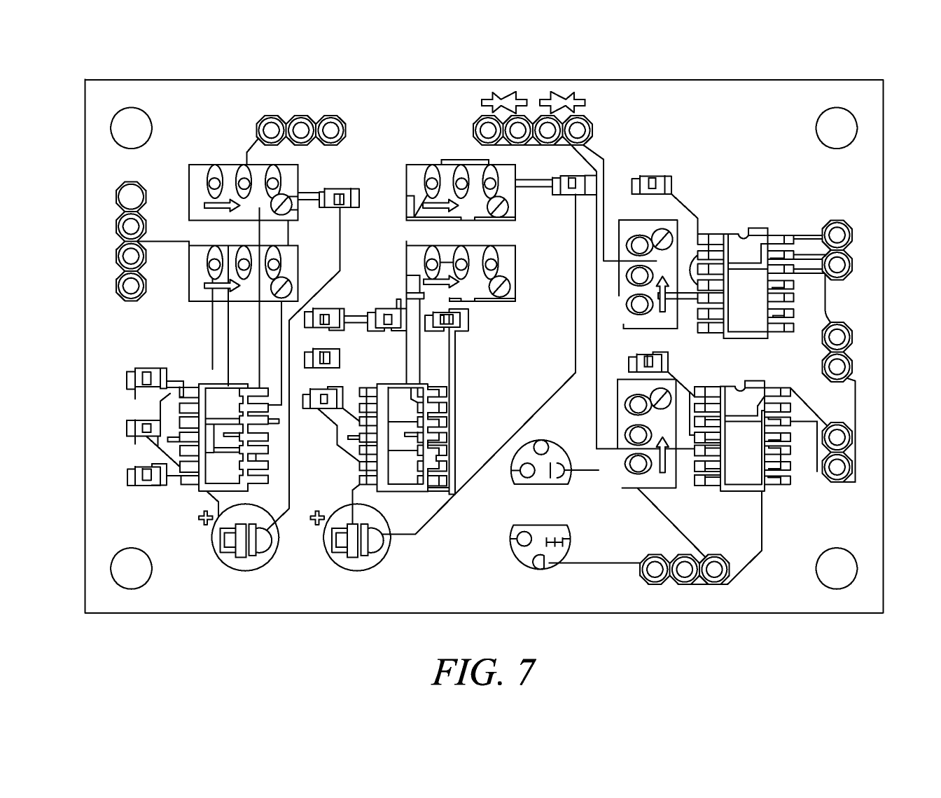

FIG. 7 shows a printed circuit board of 2-channel signal amplifier for the wireless optical photoplethysmographic knuckle motion sensor, according to an example embodiment. FIG. 7 correlates to the circuit diagram shown in FIG. 6A above.

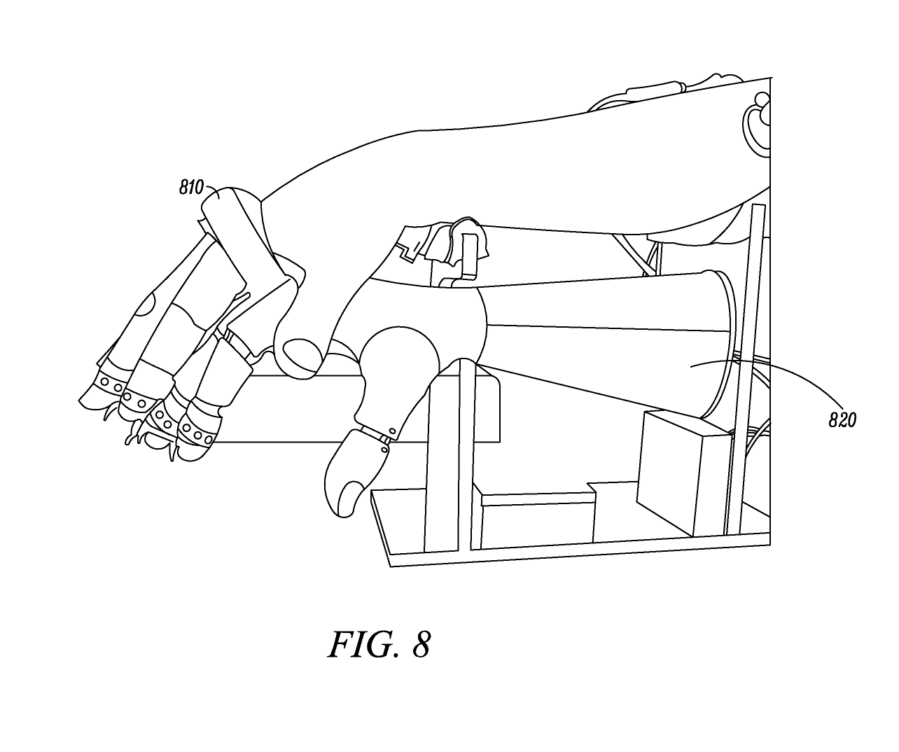

FIG. 8 shows an assembly of the wireless optical photoplethysmographic knuckle motion sensor 810 installed for wireless finger motion sensor with robotic rehabilitation device 820, according to an example embodiment.

Discussed above is a photoplethysmographic sensor for a knuckle joint. It is further contemplated that this technology could be adapted and used on other joints in a human or other animal.

A computer or other processor can be used to store data and tables to correlate the positions of bones near a joint to the intensity of light reflected from the joint. For example, data related to light reflected from one or more knuckles can be stored in a database in a computer. The computer can be programmed to relate one or more subsequent measurements from a joint to various joint or bone positions or signatures associated with motions that involve the joint and surrounding body portions.

A machine-readable medium provides instructions to a machine, such as a computer or microprocessor. The computer generally includes a personal computer, a network that includes computing elements, handheld devices that include microprocessors and the like. When executed by the machine the instructions cause the machine to perform operations including measuring an amount of reflected light from a joint when bones near the joint are in a plurality of positions, and associating the amount of reflected light from a joint to a position the one or more plurality of positions. The instructions will also cause the machine to determine a position of the bones near a joint based on subsequent measures of an amount of reflected light from a joint. In another embodiment, the instructions, when executed by the machine, cause the machine to perform operations further including determining a motion from a plurality of subsequent measures of reflected light of the bones near a joint. The computer readable media includes storage devices such as disk drives, solid state memories, or the like. In addition, media contemplates an internet connection to such storage devices. When a computing device or microprocessor runs the instruction set it is generally termed a specialized machine.

This has been a detailed description of some exemplary embodiments of the invention(s) contained within the disclosed subject matter. Such invention(s) may be referred to, individually and/or collectively, herein by the term “invention” merely for convenience and without intending to limit the scope of this application to any single invention or inventive concept if more than one is in fact disclosed. The detailed description refers to the accompanying drawings that form a part hereof and which shows by way of illustration, but not of limitation, some specific embodiments of the invention, including a preferred embodiment. These embodiments are described in sufficient detail to enable those of ordinary skill in the art to understand and implement the inventive subject matter. Other embodiments may be utilized and changes may be made without departing from the scope of the inventive subject matter. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed:

1. A method for measuring movement about a joint comprising:
positioning a photoplethysmographic sensor at a joint;
emitting light from the photoplethysmographic sensor to the joint; and
measuring an amount of reflected light from the joint.

2. The method of claim 1 wherein the photoplethysmographic sensor includes an optical sensor.

3. The method of claim 1 wherein the amount of reflected light is represented by a signal.

4. The method of claim 1 wherein the amount of reflected light over time from a particular joint produces a signature signal correlating to movement of one or more bones near the joint.

5. The method of claim 1 further comprising transmitting the signal wirelessly.

6. The method of claim 1 wherein the joint is a knuckle.

7. The method of claim 1 wherein a plurality of joints are provided with photoplethysmographic sensors and at least two of the plurality of joints are measured substantially simultaneously.

8. The method of claim 8 wherein the combination of a measurement from a first joint and a second joint depicts a movement.

9. The method of claim 8 wherein the combination of a measurements over a time from a first joint and a second joint depicts a movement of bones near the joint.

10. An apparatus for measuring movement comprising;
a strap for holding a photoplethysmographic sensor proximate a joint, the photoplethysmographic sensor further including;
a light emitter for emitting light into a joint region;
a light detector for receiving reflected light from the joint region, the light detector producing a signal in response to an amount of reflected light received at the light detector; and
a processing device for associating the amount of light reflected and received at the light detector to a position of bones near the joint.

11. The apparatus of claim 10 wherein the strap supports a plurality of photoplethysmographic sensors.

12. The apparatus of claim 10 wherein the strap supports a plurality of photoplethysmographic sensors, the strap allowing for adjustment of the position of the photoplethysmographic sensors with respect to the strap.

13. The apparatus of claim 10 wherein a spacing between a plurality of photoplethysmographic sensors can be adjusted to position of the photoplethysmographic sensors near a plurality of joints to be measured.

14. The apparatus of claim 10 wherein a spacing between a plurality of photoplethysmographic sensors can be adjusted to position of the photoplethysmographic sensors near a plurality of knuckles to be measured.

15. The apparatus of claim 10 wherein the light emitter for emitting light into a joint region includes a light emitting diode (LED).

16. The apparatus of claim 10 wherein the light detector for measuring reflected light from a joint region includes a photosensor.

17. A machine-readable medium providing instructions that, when executed by a machine, cause the machine to perform operations comprising:
measuring an amount of reflected light from a joint when bones near the joint are in a plurality of positions;
associating the amount of reflected light from a joint to a position the one or more plurality of positions; and
determining a position of the bones near a joint based on subsequent measures of an amount of reflected light from a joint.

18. The machine-readable medium of claim 17 for providing instructions that, when executed by a machine, cause the machine to perform operations further comprising determining a motion from a plurality of subsequent measures of reflected light of the bones near a joint.
